(19) United States
(12) Patent Application Publication
(10) Pub. No.: US 2024/0371060 A1
(43) Pub. Date: Nov. 7, 2024

(54) METHOD AND DEVICE FOR SUPERIMPOSING ON OBJECTS IN DIGITAL IMAGES

(71) Applicant: KARL STORZ SE & Co. KG, Tuttlingen (DE)

(72) Inventors: Lukas BUSCHLE, Tuttlingen (DE); Simon HAAG, Tuttlingen (DE); Jasmin KEUSER, Tuttlingen (DE); Bernd STEINKE, Tuttlingen (DE)

(73) Assignee: KARL STORZ SE & Co. KG, Tuttlingen (DE)

(21) Appl. No.: 18/652,873

(22) Filed: May 2, 2024

(30) Foreign Application Priority Data
May 3, 2023 (DE) 10 2023 111 479.4

(51) Int. Cl. G06T 11/60; A61B 1/00; A61B 90/00; G06T 7/11; G06T 7/194

(52) U.S. Cl. CPC G06T 11/60; A61B 1/00009; A61B 1/0005; A61B 90/361; G06T 7/11; G06T 7/194; A61B 2090/364; A61B 2090/373; G06T 2200/04; G06T 2207/10012; G06T 2207/10068; G06T 2207/30004; G06T 2210/41; G06T 2210/62

(57) ABSTRACT

The disclosure relates to a method for the at least partial optical overlay on at least one piece of information in the foreground of a digital image of an observed region, recorded by a medical imaging device, with pieces of image information corresponding to the region in the field of view of the medical imaging device concealed by the at least one piece of information.

### CROSS-REFERENCE TO RELATED APPLICATIONS

This application claims priority of German Patent Application No. 10 2023 111 479.4 filed on May 3, 2023, the contents of which are incorporated herein.

### TECHNICAL FIELD

The present disclosure relates to a method and a device for superimposing on objects in the foreground of digital images with pieces of image information corresponding to the respective background.

### BACKGROUND

In endoscopy, endoscopes are used to visualize the work region in the interior of a patient, and one or more endoscopic instruments are used to perform the corresponding medical procedure. As a rule, the instruments are introduced at a very acute angle relative to the endoscope in the process, whereby the instrument shaft is very close to the endoscope and accordingly visualized very large in the image.

Objects such as endoscopic instruments close to the endoscope take up a large part of the image, and this may lead to the user, for instance a surgeon, seeing less of the actual work region and important regions possibly being concealed by the instrument. In specific applications, for example cystoscopy, the angle between instrument and endoscope reduces to 0°; in this case, the instrument and endoscope are moved along the same axis, whereby the overlay on the work region with the instrument can be even more pronounced in the visualization.

In addition, instruments or parts of instruments such as instrument shafts located close to the optical unit and hence also located in the effective region of the light source might reflect light and consequently lead to reflections in the image. These reflections can be detrimental to the overall impression of the image, for example by virtue of leading to a reduction in the brightness of the background or a reduction in the contrast due to stray light.

Accordingly, it is an object of the present disclosure to at least partly overcome the limitations and deficiencies of the methods and systems known from the prior art. In particular, it is an object of the present disclosure to make available suitable methods and systems by means of which it is possible to recognize certain instruments, parts of instruments, and/or other objects or pieces of information which might be bothersome or irrelevant in the endoscopic field of view and overlay image information from the image background thereon in order to thus represent these virtually transparently, with the result that the user can recognize the relevant structures or pieces of information in the background. At the same time, users of the method or the systems should be able to recognize the current pieces of information which precisely correspond to the real endoscopic image and the pieces of information which are slightly delayed or less certain in terms of detail but e.g. allow orientation in the work region.

### SUMMARY

Accordingly, the present disclosure discloses methods for the at least partial optical overlay on at least one piece of information in the foreground of a digital image, in particular an endoscopic image. In this context, the method according to the disclosure comprises the use of a medical imaging device to record at least one digital image in a field of view of the medical imaging device; the use of an image processing device to segment at least one image region in the recorded at least one image; the use of the image processing device to select the at least one image region in the at least one digital image which corresponds to the at least one piece of information; the use of the image processing device to overlay on the image region in the at least one image with image information; and the use of a display device to display the at least one image together with the overlaid-upon image region. The method according to the disclosure is characterized in that the image information used for the overlay corresponds to the region in the field of view of the medical imaging device concealed by the at least one piece of information. In embodiments of the present disclosure, the medical imaging device is an endoscope camera, in particular a stereo endoscope camera, or an exoscope camera.

A piece of information within the sense of the present disclosure can be any type of pictorial elements in an image. In particular, pieces of information can be image representations of objects such as instruments or parts of instruments or else optical effects such as reflections of or superimpositions on individual pixels or groups of pixels. Thus, in particular, saturated regions, i.e. maximally white regions, e.g. as a result of reflections, overexposed edge regions, individual frames or regions of individual frames superimposed upon by laser or else individual lines and partial lines superimposed upon by laser in the case of rolling shutter-based sensors are also pieces of information within the sense of the present disclosure.

Segmentation within the meaning of the present disclosure is the acquisition and generation of contextually connected regions by combining neighboring pixels in accordance with certain homogeneity criteria. In principle, segmentation can be implemented in different ways within the scope of the present disclosure, in particular by means of pixel-, edge- and region-oriented methods. In addition, a distinction can be made between model-based methods, in which a certain shape of the objects is assumed, and texture-based methods, in which an internal homogenous structure of the object is also taken into account. In principle, all of the aforementioned methods can be used within the scope of the present disclosure, even in combination. In particular, segmentation can also be implemented with the aid of artificial intelligence algorithms. In this context, it is particularly advantageous for the algorithm to have been trained using objects which might be contained in the images from the subsequent application of the methods. Thus, when endoscopes are used during medical procedures, it is advantageous if the algorithm can recognize the various instruments used during a medical procedure in order thus to improve the quality of the segmentation. Optionally, this recognition can be manual, semi-automatic or automatic and can optionally also be implemented using artificial intelligence algorithms. Optionally, segmentation and recognition can be implemented at the same time. In particular, use can be made of so-called multiclass segmentation algorithms, in which the segmented image regions are assigned to certain classes. In the example of the endoscopic, medical procedure, use could be made of for example the following classes: tissue, work element (i.e. the tool region of an endoscopic instrument), and instrument shaft. Consequently, segmented image regions can be immediately assigned and recognized, and this assignment can be used by the method according to the disclosure for overlaying on and transparently depicting certain image regions, for example the instrument shaft.

In embodiments of the method according to the disclosure, the image information for the overlay can be adapted in terms of perspective to the image region which should be overlaid upon. To this end, structures, contrasts, and color regions in particular can be captured and brought into correspondence.

In embodiments of the method according to the disclosure, the image information can be extracted from at least one image which was recorded before the at least one digital image and contains the image information corresponding to the region in the at least one digital image concealed by the at least one piece of information.

In some embodiments, the image information can be created by the image processing device with the aid of generative image processing methods based on artificial intelligence. In this case, artificial intelligence-based algorithms, for example InPainting or OutPainting, can be used to generate optically suitable pieces of artificial image information. These methods are carried out in particular if no adequate pieces of image information are available for the image region to be overlaid upon. These methods are based on a successive continuation of the pieces of edge information into the missing region, e.g. the image interior, in order thus to fill the gap in the depiction that has arisen by the coverage by the object. For example, the algorithms used extend lines with the same grayscale values at the edge iteratively into the region to be filled. Subsequently, colors can be incorporated in the image region to be filled. The object of these techniques in general image processing is to create a modified image in such a way that changes are not noticeable to the observer; however, since this is artificial image information as a matter of principle, this image information is fundamentally unreliable. Accordingly, the inserted pieces of image information in the depiction should be marked as uncertain or unreliable for the user.

In some embodiments, the image information can be extracted from at least one image recorded from a different perspective. In some embodiments, the at least one image recorded from a different perspective can be recorded by a second image processing device. In some embodiments, the image recorded from a different perspective can be recorded following a movement of the medical imaging device, wherein the movement can preferably be performed by a motorized system, for example by a robotic arm, in order thus to obtain pieces of information with regards to the movement and give consideration thereto in a possible subsequent image analysis. In embodiments of the present disclosure, the movement can be implemented perpendicular to the main orientation of the pieces of information.

In some, preferred embodiments, the medical imaging device may comprise two objectives arranged next to one another and respectively being able to be used for recording a right and a left image, the at least one digital image being able to be a three-dimensional image. In this context, it is possible to use in particular camera systems with either an axially parallel alignment or a convergent alignment. On account of the different perspectives of the two cameras, pieces of information such as objects in the vicinity of the cameras for example are imaged at different positions in the respective image planes. Accordingly, different regions of the observation region in the background of the image are covered in the left and right images by the information in the foreground. Thus, image information, as two-dimensional information, can be extracted from the left image and/or the right image. If a camera system with a convergent alignment is used, it is advantageous to calibrate the images in order to facilitate the assignment of objects in the respective images. The calibration is preferably implemented according to the principles of epipolar geometry. Provided pieces of image information regarding concealed regions cannot be extracted from the left image and/or the right image either, the corresponding pieces of image information can be extracted and/or created according to other methods, for example the methods described above, in preferred embodiments.

In embodiments of the present disclosure, the overlaid-upon image region can be presented transparently, partly transparently, and/or as a contour. In some embodiments, the type of presentation of the overlaid-upon image region may also depend on the reliability of the image information used. In some embodiments, the reliability of the pieces of image information used can be determined by way of a plausibility check based on predefined metrics. Accordingly, the reliability of pieces of image information extracted from stereo image pairs can be categorized as reliable since these pieces of image information were captured at the same time as the image and consequently represent the actual situation at the moment the image was recorded. Pieces of image information extracted from images recorded earlier can be categorized as increasingly uncertain, depending on the age of the images and/or also depending on the movement of the observation region or the camera. Artificially generated pieces of image information can be categorized as uncertain. In embodiments of the present disclosure, the reliability of the overlay can be determined by the comparison of the pieces of overlaid image information with the at least one image.

In embodiments of the disclosure, the depiction of the overlay can reflect the reliability of the pieces of image information. Accordingly, introduced image information in the presentation can be marked as uncertain or unreliable for the user, for example by desaturation or else graying out. Consequently, the user can recognize that, depending on the presentation, the inserted image information serves for example only for the geometric spatial orientation and for the purpose of reducing the distraction due to e.g. shafts, and cannot be used for any informative, diagnostic or even therapeutic purpose.

In aspects, the disclosure relates to a system containing at least one medical imaging device, at least one image processing device, and at least one display device. In some embodiments, the system can be configured to be able to carry out an above-described method. In some embodiments, the image processing device implements a discriminator in order to determine the reliability of pieces of image information generated by generative image processing methods based on artificial intelligence.

One aspect of the disclosure relates to a computer program having instructions which, if the program can be executed by a computer, cause the computer to carry out one of the above-described methods.

One aspect of the disclosure relates to a computer-readable storage medium containing instructions which, when executed by a computer, cause the computer to carry out the methods of points 1 to 14.

### BRIEF DESCRIPTION OF THE DRAWINGS

Further aspects of the disclosure will become clear from the following description of preferred exemplary embodiments and from the attached figures, in which:

FIGS. 1a, 1b show the recording of stereo image pairs using cameras aligned in axially parallel fashion;

FIGS. 2a, 2b show an axially parallel camera system and the image representation of a point in the corresponding image planes;

FIGS. 3a, 3b, 3c show a convergent camera system, the imaging of a point in the corresponding image planes, and a calibrated image pair;

FIG. 4 shows the course of the method according to the disclosure;

FIG. 5 shows the course of the alternative methods for determining image information;

FIG. 6 shows the course of the method according to the disclosure for overlay on the basis of stereo image pairs;

FIGS. 7a, 7b show a stereo image pair;

FIGS. 7c, 7d show the stereo image pair following the calibration;

FIGS. 8a, 8b show depth maps of the calibrated stereo images;

FIGS. 8c, 8d show the segmentation of the depth maps; and

FIGS. 9a, 9b show the overlay of the corresponding image information.

### DETAILED DESCRIPTION OF PREFERRED EMBODIMENTS

The disclosure is based on augmenting, possibly with a perspective correction, background tissue (still) available from different spatial and/or temporal perspectives but currently concealed in the image by pieces of information, for example instruments or else reflections, and it involves the appropriate calculation of the probability corresponding to the respective method that this augmentation corresponds to the reality and the appropriate presentation of this probability.

A stereographic representation of observation regions requires two images of the same observation region recorded at two different positions that are spaced apart from one another by a certain distance—the base. Stereo recordings can be generated in different ways. If only a single camera is used, successive images of a sequence are taken as stereo images; as it were, a stereo camera system is simulated by moving the camera. In the case of fixed base stereo cameras, two cameras are arranged at a fixed distance from one another, the base length is specified, and the cameras produce stereo image pairs in an axially parallel arrangement. In this case, the recording directions are normal to the baseline and parallel to one another, facilitating the stereo analysis.

Accordingly, various stereo algorithms assume this geometric arrangement of the cameras. Camera systems with a convergent arrangement are used in an alternative. This may be intentional or else for system- or production-related reasons; however, this convergence can be compensated in a postprocessing step by a virtual rotation of the images. This process is referred to as (epipolar) straightening or rectification, or else as calibration.

The object of stereo camera systems is that of providing, either virtually or actually, images in an axially parallel arrangement in which the recording directions are normal to the baseline and parallel to one another, facilitating subsequent stereo analysis.

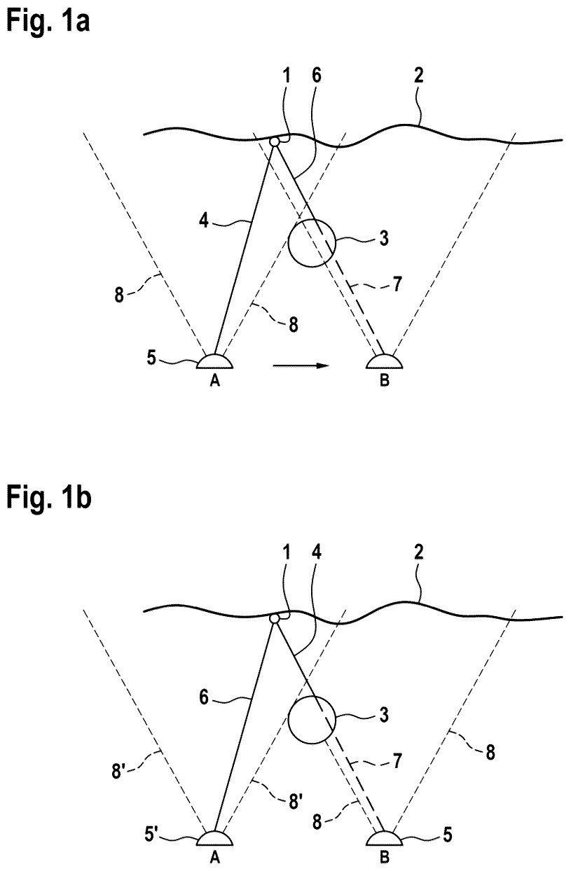

FIG. 1a shows a scenario in which a first image of an observation region is recorded by means of a single camera 5 in position A. The observation region comprises a background 2 and an object 1. The lines 8 describe the viewing angle and hence the field of view of the camera, and the line 4 represents the ray for imaging the object 1. For the given viewing angle of the camera 5, the object 3 is not in the field of view of the camera 5 when the camera is in its position A. Subsequently, the camera is moved to position B and a second image of the observation region is recorded. In position B, the object 3 enters the field of view of the camera and thus conceals portions of the actual observation region in the image representation, specifically inter alia the object 1, as indicated by the depiction of the ray 6, 7, and parts of the background 2.

FIG. 1b schematically shows a corresponding scenario for recording two stereo images of an object 1 in front of a background 2 using two cameras 5, 5′ in axially parallel arrangement, with an object 3 being arranged in the foreground. In this case, the lines 8 and 8′ describe the viewing angles of the cameras 5, 5′, respectively, and the lines 6 and 7 represent the rays for imaging the object 1 on the respective cameras 5, 5′. The ray 6 can reach the camera 5′ unimpeded, with the ray 4 being blocked by the object; the hypothetical continuation 7 as far as the camera 5 thus does not lead to the object 1 being imaged on the camera 5. Thus, a portion of the observation region is concealed by the object 3 in the overlay of the two stereo images serving for a three-dimensional representation of the observation region comprising the object 1 and the background 2.

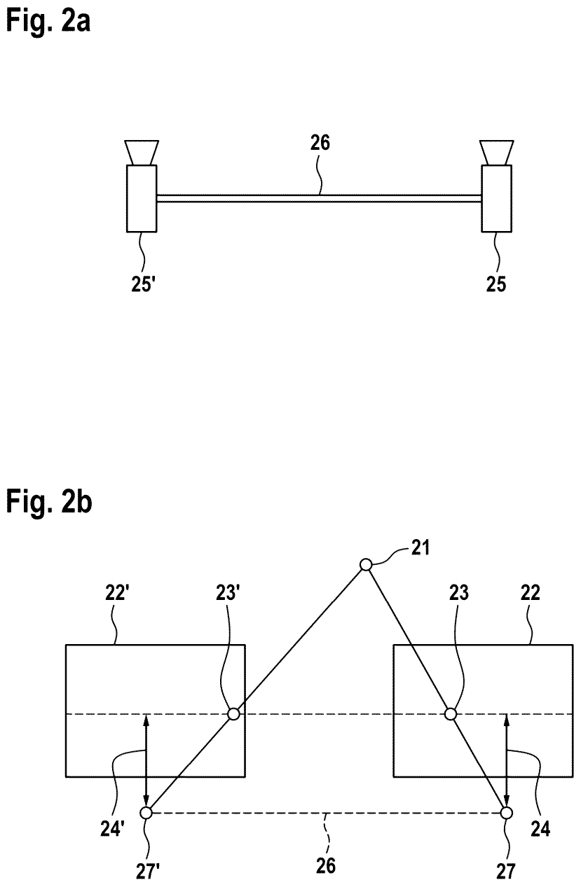

FIGS. 2a and 2b show a schematic illustration of an axially parallel stereo system for recording stereo image pairs. The system is distinguished by two cameras 25, 25′ which are arranged with a horizontal displacement equal to the base length 26; the image planes 22, 22′ of the cameras 25, 25′—and hence their coordinate systems—are not twisted relative to one another. FIG. 2b depicts the front view of the axially parallel system of FIG. 2a. The image planes 22, 22′ are in parallel and the two optical centers 27, 27′ are displaced horizontally by the base length 26. The focal length 24, 24′ defines the distance of the two foci from their image planes 22, 22′ and is assumed to be identical in the illustration for the two cameras. Thus, an object 21 is projected by the two optical centers on two image points 23, 23′ in the respective image planes 22, 22′. Since the image lines in the respective digital images are identical in the case of an axially parallel stereo system, the different perspectives of the cameras with regards to the object 21 lead only to a horizontal offset of the image points 23, 23′ in the image representation thereof. This difference is referred to as disparity and represents the relative displacement of the image representation of the object 21. If objects are situated between the cameras and the structures to be imaged, the optical image representation can be interfered with or overlaid, as described in the context of FIG. 1.

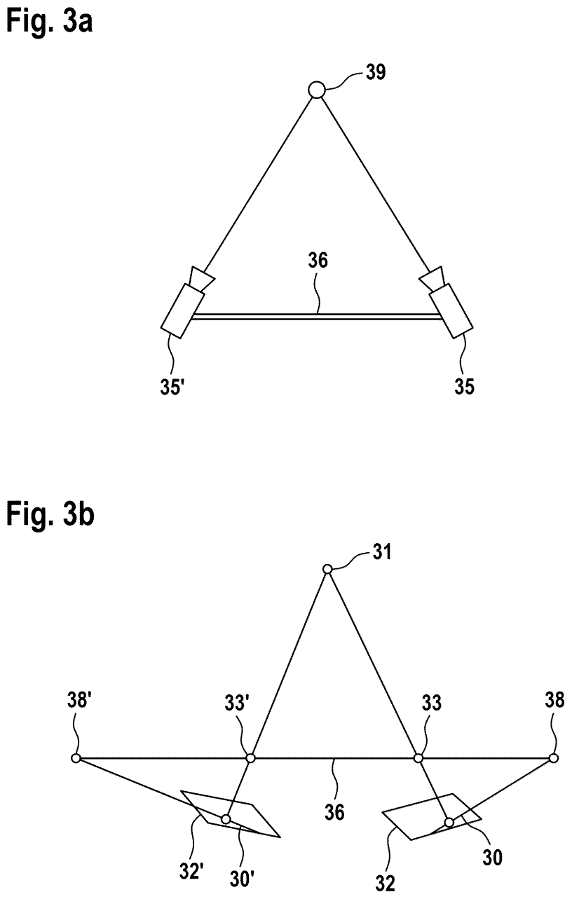

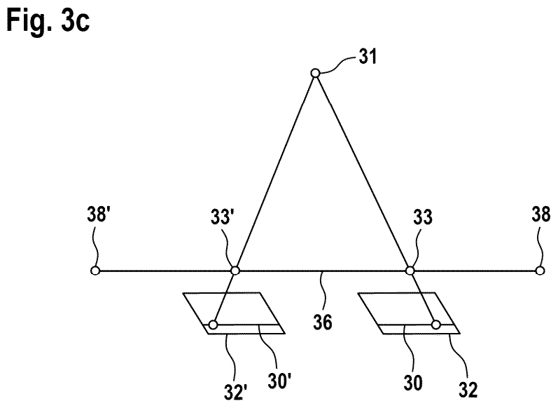

FIGS. 3a to 3c show a schematic illustration of a stereo system with a convergent arrangement of the cameras 35, 35′ which are displaced by the base length 36; the optical axes of the cameras 35, 35′ are aligned on a convergence point 39. Accordingly—and unlike the case of the axially parallel system in FIGS. 2a and 2b—the two cameras 35, 35′ are not only displaced horizontally but also twisted relative to one another. Such twists in relation to the alignment of the camera may be desired or else be system- or else production-related deviations from an axially parallel alignment. The mathematical model of epipolar geometry, which describes the geometric relationships between different camera images of the same object, allows the dependence between corresponding image points—i.e. the points which an individual object point creates in the two camera images—to be described.

To enable the assignment of pieces of information between the two image planes or between the images recorded by the two cameras 35, 35′, the respective images should be rectified. The object of rectification with regards to a stereo image pair lies in the simulation of a perfect stereo camera system, in which the optical axes run in parallel and the image planes are coplanar.

In epipolar geometry, the so-called epipolar plane is defined by the projection centers of the two cameras 33, 33′ and an image point 31. The epipolar lines 30, 30′ arise from the intersection of the epipolar plane with the image planes 32, 32′ of the cameras 33, 33′. All epipolar lines of an image plane 32, 32′ intersect at the epipoles 38, 38′. A different epipolar plane, and hence likewise different epipolar lines, arise if the image point 31 is changed. FIG. 3b schematically shows the course of the epipolar lines 30, 30′ transversely across the image areas 32, 32′ of the cameras 33, 33′ in convergent arrangement.

As a result of rectification, the convergent camera arrangement is now converted mathematically into an axially parallel stereo system. As depicted schematically in FIG. 3c, the epipolar lines 30, 30′ are aligned parallel to one another as a result of the rectification, whereby the epipoles 38, 38′ move to infinity. The image representation of corresponding image points in the images now is always located on corresponding lines in the corresponding images. Following rectification, the search for correspondence reduces to the search between a line in the left image and the corresponding line in the right image.

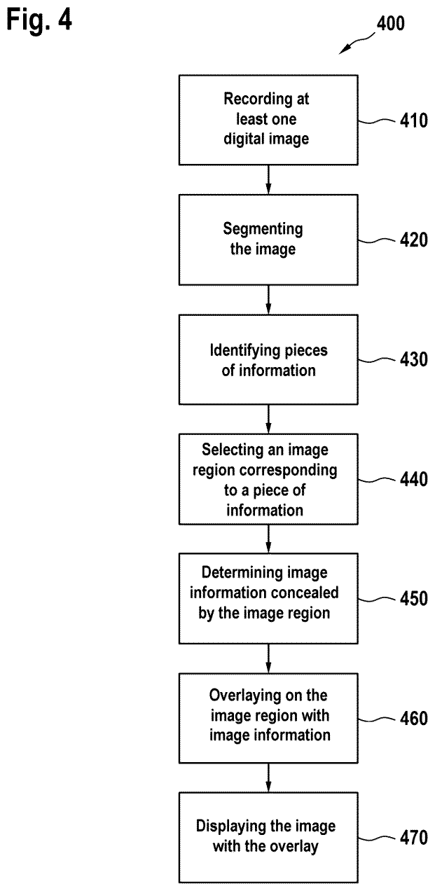

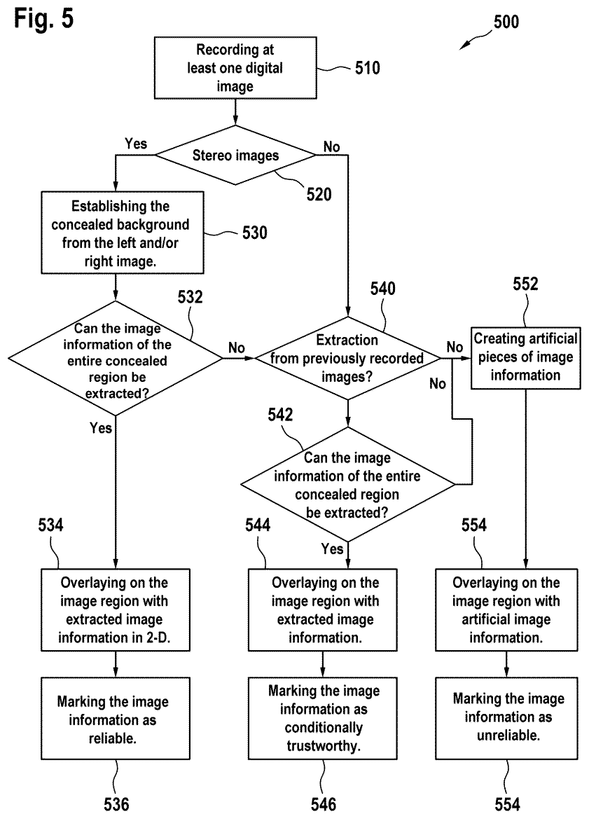

FIG. 4 describes the main features of the method 400 according to the disclosure. In a first step 410 of the method, a digital image is recorded by means of a medical imaging device, for example an endoscope camera. Depending on the type of image material recorded, for example stereo image pairs, single images, video frames, etc., individual method steps may differ, as described in detail in the context of FIG. 5. However, the basic method steps described in the context of FIG. 4 find largely universal application.

The image is segmented in subsequent step 420, which is to say contextually connected regions are created by combining adjacent pixels in accordance with a certain homogeneity criterion. In principle, segmentation can be implemented in different ways within the scope of the present disclosure, in particular by means of pixel-, edge- and region-oriented methods. In addition, a distinction can be made between model-based methods, in which a certain shape of the objects is assumed, and texture-based methods, in which an internal homogenous structure of the object is also taken into account. In principle, all of the aforementioned methods can be used within the scope of the present disclosure, even in combination. In particular, segmentation can also be implemented with the aid of artificial intelligence algorithms. In this context, it is particularly advantageous for the algorithm to have been trained using objects which might be contained in the images from the subsequent application of the methods. Thus, when endoscopes are used during medical procedures, it is advantageous if the algorithm can recognize the various instruments used during a medical procedure in order thus to improve the quality of the segmentation.

Subsequently, certain pieces of information which can be assigned to segmented regions can be recognized and optionally identified in step 430 of the method. Optionally, this recognition can be manual, semi-automatic or automatic and can optionally also be implemented using artificial intelligence algorithms. Optionally, segmentation and recognition can be implemented at the same time.

In particular, use can be made of so-called multiclass segmentation algorithms, in which the segmented image regions are assigned to certain classes. In the example of the endoscopic, medical procedure, use could be made of for example the following classes: tissue, work element (i.e. the tool region of an endoscopic instrument), and instrument shaft. Consequently, segmented image regions can be immediately assigned and recognized, and this assignment can be used by the method according to the disclosure to overlay upon and hence transparently depict certain image regions, for example the instrument shaft.

Subsequently, an image region corresponding to a certain identified piece of information can be selected in step 440 of the method as the image region which is intended to be overlaid upon with image information in the subsequent method steps. This selection can be made manually, i.e. by the user of the method, or else automatically, for example on the basis of the recognition. Thus, again using the example of endoscopes in medical procedures, a certain instrument or a part of an instrument such as an instrument shaft can be selected automatically for a subsequent overlay thereon.

Subsequently, the piece of image information in the observation region concealed by the object in the foreground recognized in the preceding steps is ascertained in step 450. According to the disclosure, different methods can be used for ascertaining the image information. Thus, according to the disclosure, the image information can be extracted from a stereo image pair provided the concealed object is arranged in such a way that corresponding portions of the observation region are in each case only covered in one of the two images. This method is based on the different spatial perspective of the two cameras as these, as described above, are at least arranged with horizontal spacing, and optionally also in convergent fashion, and consequently differ in terms of their fields of view. Since the pieces of image information extracted thus were recorded at the same time, the content corresponds exactly to what was concealed by the object in the foreground; thus, there is no uncertainty in respect of the reliability of the content. Details in relation to this method are described in the context of FIG. 5.

In an alternative or in addition, for example if it is not possible to extract the complete image information about the concealed region from the stereo image pair, image information can be extracted from a different temporal perspective, i.e. from images recorded in advance. In many cases, the observation region is visualized in the form of video frames, i.e. temporally successive images of the observation region. Depending on the situation there might optionally be older images in which the region of the observation region concealed in the current image is not concealed. On the one hand, this may be traced back to a movement of the camera. For example, the region situated in the image center in the current image, which is concealed by an object, may accordingly have been at the image edge in previous images before the camera moved. On the other hand, or in addition, the concealing object might not yet have been in the field of view of the camera in previous images. Using the example of the endoscopic medical procedure, this might for example arise due to the introduction of an instrument. Given the time lapse between the recording of the image used for extracting the image information and the current image into which the image information is intended to be inserted, the reliability of the content may be afflicted by some uncertainty dependent on the age of the image from which the image information is extracted. Details in relation to this method are described in the context of FIG. 5.

In an alternative or in addition, image information can be created artificially, for example if it proved impossible to extract the entire image information about the concealed region from the stereo image pair or from previous images. As described in detail in the context of FIG. 5, artificial intelligence algorithms, for example InPaint algorithms, can be used to this end. However, since the pieces of artificially generated image information are purely synthetic data that may fit optically into the image but do not reproduce any real image information, this image information is uncertain with regards to the reliability of the content as a matter of principle. Details in relation to this method are described in the context of FIG. 5.

Once the image information was extracted and/or generated, as described above, the image region selected post segmentation is overlaid upon with this image information in the next step 460, wherein perspective adaptations of the image information can be carried out where necessary. As a result of the overlay, the concealing object is presented virtually transparently. In this case, an indication for the reliability of the image information used for the overlay can be provided by the presentation of the overlay. Thus, the introduced image information in the presentation can be marked as uncertain or unreliable for the user, for example by desaturation or else graying out. Consequently, the user can recognize that the inserted pieces of image information serve only for the geometric spatial orientation and for the purpose of reducing the distraction due to e.g. shafts, and cannot be used for any informative, diagnostic or even therapeutic purpose.

FIG. 5 describes the method 500 according to the disclosure using the various options for augmenting image regions concealed in a current image using appropriate image information. In principle, the individual options described in FIG. 5, especially the extraction of image information from stereo image pairs or from images recorded from a different perspective using a further medical imaging device, the extraction of image information from previous images, or the generation of artificial image information can be applied independently of one another or else in combination with one another. In embodiments of the present disclosure, the two medical imaging devices may together represent a stereo camera, preferably a stereo endoscope camera.

At the start of the method 500, at least one digital image is recorded in a first step 520 by means of an appropriate medical imaging device, for example an endoscope camera. Depending on the medical imaging device, this may be a single image or, in the case of a stereo camera such as a stereo endoscope for example, this may be a stereo image pair, i.e. partly overlapping images recorded at the same time from different perspectives. In the next step 520 of the method, a corresponding distinction is made as to whether this pertains to a single image or a stereo image pair.

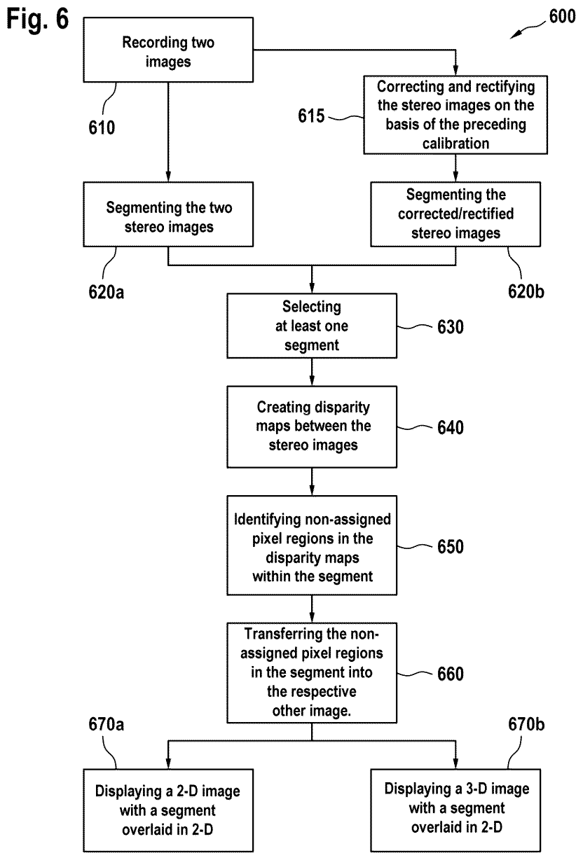

In the case of a single image, the method continues with step 540, which is described in detail below. In the case of a stereo image pair, the method is continued with step 530, in which the images are segmented, and the concealed background in the two images is established. Details in relation to this method step are described in FIG. 6. Based on the outcome of this method step 530, a subsequent check is performed in step 532 as to whether pieces of image information can be extracted for the entire concealed region from the stereo image pair. If the concealing object is too large in the image recording, too close to the camera, and/or too close to the background, then the disparity becomes too small, and the concealed image information cannot be fully extracted from the stereo image pair. In this case, either the concealing object cannot be overlaid upon in its entirety or the image information can be obtained differently, for example by extracting it from previous frames or generating it using artificial intelligence algorithms. Corresponding method steps 540, 552 are described in detail below.

Should the verification in step 532 yield that the image information of the entire concealed region can be extracted from the stereo image pair, the extracted image information is inserted into the corresponding segment in step 534; thus, the concealing object is overlaid upon and consequently depicted transparently. Since the used image information is taken from only one of the two stereo images in each case (or is composed from pieces of information from the left and the right stereo image), the presentation of this region is two-dimensional. The object made “invisible” by the method can be presented in different ways in the representation. Accordingly, the object can be rendered completely transparent, i.e. be masked in full. In an alternative, the outline of the object can be depicted, or the object can be presented partly transparently, for example by way of alpha-blending.

If required, the image information can be adapted in terms of perspective to the segment in which the information should be inserted.

Since the image information is real information recorded at the same time, there is no uncertainty pertaining to the content. Accordingly, the image information can be marked as certain in the display on an output device such as a monitor or a pair of smartglasses within the scope of an optional step 536. Various methods can be used to mark the reliability in this way. For example, as the reliability of content decreases, the inserted image information can be progressively desaturated or partially grayed out.

The method described in steps 540 to 546 can be carried out if, as described above, stereo image pairs are not available or the entire image information of the concealed region cannot be extracted. In this context, a check is carried out in step 540 as to whether at least one previously recorded image is available. In this context, use is made in particular of images recorded prior to the movement of the medical imaging device from a different perspective or before the concealing object entered the field of view. For example, following a change in the perspective of the camera and/or position of the object, the image information of the regions concealed in the current image by the object might optionally still be extracted from a previous frame of a video recording recorded by means of an endoscope camera. In particular, in this case the system can be configured such that appropriate older images are stored or kept available for a use according to the disclosure.

The alignment of the pieces of image information or images is decisive for overlaying on image regions in an image with pieces of image information extracted from an older image. In this case, the movement can be determined and reproduced by way of so-called optical flow algorithms, for example. Movement corrections can be performed on the basis thereof.

In addition to that or alternatively, various meta-parameters such as inter alia sharpness, level of fine detail, local contrast can also be used for matching with pixel accuracy. Moreover, the recording time—and hence the age of the image—can be used as an indicator for the probability that the augmented area still is acceptably current. In particular, the recording time can also be used in combination with pieces of movement information as an indicator for the probability that the augmented area still is acceptably current. The image information used for the superimposition can be displayed in accordance with the determined reliability of the information; thus, the information can for example be depicted in desaturated or grayed-out fashion.

In embodiments of the disclosure, techniques known as burst imaging can be employed, utilizing movement-corrected and regionally realized “long-term exposures” to create noise-reduced brighter textures with enhanced dynamics and diminished motion unsharpness even in the dark image regions using one or typically more older frames. Equally, overlaying on pieces of information such as concealing objects allows the latter to be masked. Depending on the application or configuration, concealing objects or information (such as reflections, superimposed images or image regions, etc.) thus can be superimposed upon, and this improves the image quality.

Burst imaging employs this movement-corrected and regionally realized “long-term exposure” to create noise-reduced brighter textures with enhanced dynamics and diminished motion unsharpness even in the dark image regions using one or typically more older frames.

For a good image quality, a plurality of parameters are acquired for each tile of the current frame and each tile of the older moved frames: Sharpness and level of detail in the movements should be tracked with at least pixel accuracy. Likewise, the age of the frames reduces the predictive accuracy and represents the 3rd factor for the reliability of the information. Accordingly, the positive factor of sharpness should be given a lesser weighting as the age of frames increases. Conversely, a lower level of detail is positive as there are fewer risks of errors with the respective tile. This collectively determines the reliability, to be presented, of the image information.

When the burst imaging method is applied, the quality of individual image tiles in the image pyramid can be captured for different levels of resolution and determined for weighted quality metrics. These metrics form the basis for the calculation of sharper image results in the case of disadvantageously variable light conditions, and for the prevention of errors such as incorrect alignments, at least at pixel-level accuracy.

According to the disclosure, these quality metrics can be used as set forth below for presenting a reliability: The time of recording can be significantly further in the past than what is advantageous for live image burst imaging. In the case of a live image, 1-5 frames, and up to 10 frames depending on the HDR scene, are advantageous, i.e. at 60 fps: 16.6 ms-83 ms, up to 166.6 ms in extreme cases. For the method according to the disclosure for overlaying on pieces of information, the latter are thinned out to the optically best frames on account of the quality metrics, but are for example kept for up to 1 or 5 seconds. This maximum time interval arises dynamically by way of the comparison of older tiles with then newly acquired tiles. These older tiles are depicted as possibly increasingly “outdated” at an accelerating rate but are used until newer tiles have been captured for this background position, for example as a result of movement. Alternatively, in a scene with less movement, tiles that are too old, e.g. tiles with an age>5 s, should be blurred in addition to the desaturation in order to be more identifiable and, on account of changes that are too large, in order to present edge matching in less bothersome fashion.

If, as described above, it proves impossible to extract any image information or complete image information regarding the concealed region, then artificial intelligence-based algorithms, for instance InPainting or OutPainting, can be used in step 552 to generate optically fitting pieces of artificial image information. In particular, this method is carried out when the availability of pieces of image information for the image region to be overlaid upon was determined on the basis of stereo image pairs and/or previous frames, and when residual regions were identified that can no longer be acceptably replaced, i.e. with image information classed as certain, using these methods.

Depending on the configuration, artificial intelligence-based InPainting is available to this end. The procedure is based on a successive continuation of the pieces of edge information into the image interior in order thus to fill the gap in the depiction that has arisen by the coverage by the object. For example, the algorithms used extend lines with the same grayscale values at the edge iteratively into the region to be filled. Subsequently, colors can be incorporated in the image region to be filled.

The object of these techniques in general image processing is to create a modified image in such a way that changes are not noticeable to the observer; however, since this is artificial image information as a matter of principle, this image information is fundamentally unreliable. Accordingly, the inserted pieces of image information in the depiction should be marked as uncertain or unreliable for the user. The pieces of introduced image information only serve for the geometric spatial orientation and for the purpose of reducing the distraction due to e.g. shafts, and not for any informative, diagnostic or even therapeutic purpose.

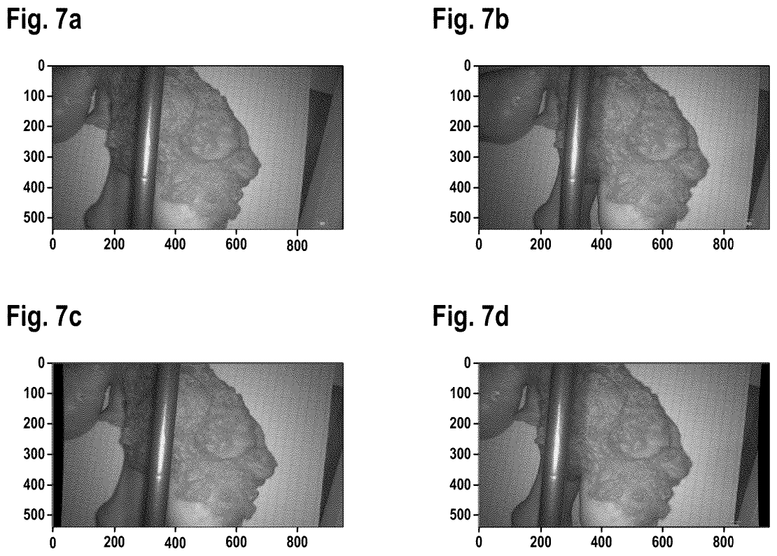

FIGS. 7 to 9 show the application of the method according to the disclosure for superimposing on an instrument shaft on the basis of stereo endoscopic recordings.

In this context, FIGS. 7a, 7b show a left and a right image recorded using a stereo endoscope. In the foreground, the images show the shaft of an instrument. A tissue model is situated on a squared background in the background of the image. It is evident that different regions of the tissue are concealed by the instrument shaft in the left and the right image. The lines of the background are slightly distorted, especially in the edge regions of the images, and this can impair an assignment of structures or pixels in the respective other image.

The two images are calibrated, i.e. corrected and rectified, to at least reduce these potential sources of errors. To this end, images of a known pattern, for example a checkerboard, are recorded in advance in a production step of the camera system. These images are used to establish various parameters such as distortion parameters, focal lengths, principal points of the camera system, and rotation and translation between the two camera sensors. The images can now be calibrated on the basis of these parameters established in advance.

FIGS. 7c, 7d show the left and right image of FIGS. 7a, 7b following a successful calibration; thus, the figures show a corrected and rectified right and left image. Accordingly, the lines of the squared background are parallel in these images; an object point in the two images is now projected on the same pixel row.

A disparity map between the left and the right image and a disparity map between the right and the left image are calculated on the basis of the corrected and rectified images. Disparity maps (or depth images) specify the disparity for each image point. Disparity maps are usually encoded as color scale images or grayscale images, with the respective color or grayscale value reflecting the disparity. To create the disparity maps, pixels corresponding between the left and right image are searched for. Within the scope of the search for correspondence, pixels in the left image are assigned to the corresponding pixels in the right image, and vice versa. The assigned image elements should be the image representation of the same 3-D point in a scene. The displacement of the pose on account of the different perspectives is referred to as binocular disparity and allows conclusions to be drawn about the depth of a pixel.

Various methods can be applied for the purpose of creating disparity maps—in particular depending on the requirements (for example quality, time, etc.) and resources available. In the context of the present disclosure, the semi-global matching algorithm by H. Hirschmüller et al. has proven its worth, with other methods known to a person skilled in the art also being possible.

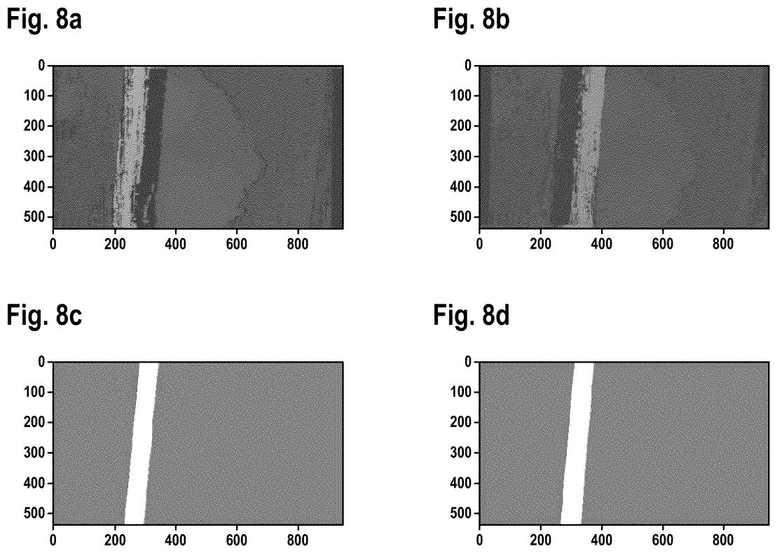

FIGS. 8a, 8b show the disparity maps based on the left and right image, with the disparity encoded as grayscale values in this case; thus, pixels at the same distance from the camera have a corresponding grayscale value.

FIGS. 8c, 8d show the segmentation of the instrument shaft in the left and right disparity map. The segmentation is implemented by means of an artificial intelligence algorithm or computer vision algorithms in the left and right image. For example, a U-Net architecture or line detector can be used to this end. It is advantageous to perform the segmentation directly on the corrected and rectified images. Subsequently, non-assigned regions which correspond to the size and shape of the segmented instrument can be identified in the disparity maps. For example, this can be performed line-by-line. The identified regions show the background concealed by the instrument such that a stereo correspondence cannot be calculated. The background regions ascertained thus can be transferred into the respective other corrected and rectified image in place of the segmented instrument shaft, and the instrument shaft thus can be superimposed upon.

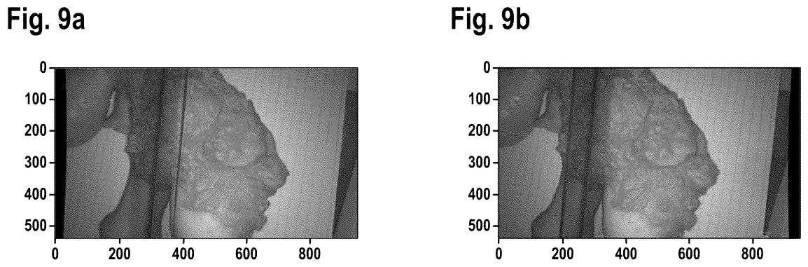

FIGS. 9a, 9b show the overlay of the corresponding image portions on the instrument shaft on the basis of the calibrated images. These images can subsequently be transformed back into the original image representation (prior to calibration, with distortion, etc.) and then be displayed to the user in a stereo image or mono image, depending on the display mode.

The scope of this disclosure includes all changes, replacements, variations, developments and modifications of the exemplary embodiments described or explained herein which would be understood by a person of average skill in the art. The scope of protection of this disclosure is not limited to the exemplary embodiments described or explained herein. Even though this disclosure comprehensively describes and explains the respective embodiments herein as specific components, elements, features, functions, operations or steps, any of these embodiments can moreover comprise any combinations or permutations of any components, elements, features, functions, operations or steps described or explained anywhere herein, which would be understood by a person of average skill in the art. A reference in the appended claims to the fact that a method or a device or a component of a device or a system for performing a specific function is adapted, set up, capable, configured, able, operative or operational, also includes this device, this system or this component, independently of whether it or this specific function is activated, switched on or enabled for as long as this device, this system or this component is adapted, set up, capable, configured, able, operative or operational to this end. Even though this disclosure describes or explains that certain embodiments provide certain advantages, certain embodiments may moreover provide none, some or all of these advantages.

1. A method for the at least partial optical overlay on at least one piece of information in the foreground of a digital image, comprising:
the use of a medical imaging device to record at least one digital image in a field of view of the medical imaging device;
the use of an image processing device to segment at least one image region in the at least one image;
the use of the image processing device to select the at least one image region in the at least one digital image which corresponds to the at least one piece of information;
the use of the image processing device to overlay on the image region in the at least one image with image information; and
the use of a display device to display at least one image together with the overlaid-upon image region;
wherein the image information corresponds to the region in the field of view of the medical imaging device concealed by the at least one piece of information.

2. The method as set forth in claim 1, wherein the medical imaging device is an endoscope camera or an exoscope camera.

3. The method as set forth in claim 1, wherein the image information is extracted from at least one image which was recorded before the at least one digital image and contains the image information corresponding to the region in the at least one digital image concealed by the at least one piece of information.

4. The method as set forth in claim 1, wherein the image information is created by the image processing device with the aid of generative image processing methods based on artificial intelligence.

5. The method as set forth in claim 1, wherein the image information is extracted from at least one image recorded from a different perspective.

6. The method as set forth in claim 5, wherein the at least one image recorded from a different perspective is recorded by a second medical imaging device.

7. The method as set forth in claim 5, wherein the image recorded from a different perspective is recorded following a movement of the medical imaging device.

8. The method as set forth in claim 7, wherein the movement of the medical imaging device is implemented by a motorized controller.

9. The method as set forth in claim 7, wherein the movement is implemented perpendicular to the main orientation of the information.

10. The method as set forth in claim 5, wherein the medical imaging device comprises two objective lenses arranged next to one another and respectively used for recording a right and a left image, the at least one digital image is a three-dimensional image, and the image information, as two-dimensional information, is extracted from the left image and/or the right image.

11. The method as set forth in claim 10, wherein image information which cannot be extracted from the left image and/or the right image is extracted in accordance with the method from claim 3 and/or generated in accordance with the method from claim 4.

12. The method as set forth in claim 1, wherein the overlaid-upon image region is presented transparently, partly transparently, and/or as a contour.

13. The method as set forth in claim 12, wherein the type of presentation of the overlaid-upon image region depends on the reliability of the image information used.

14. The method as set forth in claim 13, wherein the reliability of the image information used is determined by way of a plausibility check based on predefined metrics.

15. The method as set forth in claim 1, wherein the reliability of the overlay is determined by comparing the overlaid image information with the at least one image.

16. A system comprising at least one medical imaging device, at least one image processing device, and at least one display device, the system being configured to carry out a method according to claim 1.

17. The system as claimed in claim 16, wherein the image processing device implements a discriminator used to determine the reliability of the pieces of image information generated by generative image processing methods based on artificial intelligence.

18. A computer program comprising commands which, when the program is executed by a computer, cause the latter to carry out the methods as set forth in claim 1.

19. A computer-readable storage medium comprising commands which, when executed by a computer, cause the latter to carry out the methods as set forth in claim 1.
